(19) United States
(12) Patent Application Publication
(10) Pub. No.: US 2026/0266668 A1
(43) Pub. Date: Sep. 10, 2026

(54) DETERMINING EXHALED BREATH TEMPERATURE

(71) Applicant: DENDRO TECHNOLOGIES INC., NEW YORK, NY (US)

(72) Inventors: Adi WALLACH, NEW YORK, NY (US); Michael YAKOBY, Kfar Saba (IL)

(21) Appl. No.: 19/480,000

(22) PCT Filed: May 5, 2024
(86) PCT No.: PCT/US2024/027902

§ 371 Date: Oct. 29, 2025

Related U.S. Application Data
(60) Provisional application No. 63/464,229, filed on May 5, 2023.

(51) Int. Cl. G01K 13/024; A61B 5/097

(52) U.S. Cl. CPC G01K 13/024; A61B 5/097

(57) ABSTRACT

Disclosed are methods and devices that are, in some embodiments, useful for determining exhaled breath temperature (EBT).

### CROSS-REFERENCE TO RELATED APPLICATION

The present application is a National Phase entry under 35 U.S.C. § 371 of International Application No. PCT/US2024/027902, which claims priority from U.S. Provisional Patent Application 63/464,229, filed 5 May 2023, which is hereby incorporated by reference as if fully set-forth herein.

### FIELD

The present disclosure relates to methods and devices for determining exhaled breath temperature (EBT).

### BACKGROUND

The utility of determining exhaled breath temperature (EBT), for example for diagnosis of a pathology, has been disclosed, for example, in:
a. Piacentini, Peroni, Crestani, Zardini, Bodini, Costella, Boner in Clin Exp Allergy 2007, 37, 415-419;
b. Popov, Dunev, Kralimarkova, Kraeva, DuBuske in Resp Medicine 2007, 1010, 2044-2050;
c. Popov, Kralimarkova, Tzachev, Dunev, Dimitrov, Gill in EBT manuscript for IEEE special issue in IEEE Sensors Journal, February 2010;
d. Popov, Kralimarkova, Dimitrov in Breathe 2012, 8(3) 187-192;
e. Vermeulen, Barreto, La Penna, Martella, Biagiarelli, Villa in J Asthma 2014, 1-8;
f. Lizir, Bikov, Martinovsky, Gilffy, Losonczy and Horváth in J. Breath Res 2014, 8, 046002, 1-9;
g. Harnawan, Mariati, Assegaf in J Phys: Cond. Series 2017, 853, 012021; and
h. Tufvesson, Nilsson, Popov, Hesselstrand, Bjermer in Eur Clin Respir J 2020, 7(1), 1747014.

Methods and devices suitable for determining EBT have been disclosed, for example, in patents and patent applications such as: U.S. Pat. No. 8,323,207; US 2004/0039295; US 2010/0063409; US 2013/0030316; US 2015/0342502; US 2017/0156633; US 2018/0325421; US 2019/0175065; US 2021/0212595; as well as in EP 2745775 and WO 2009/006655.

It would be useful to have a device for determining EBT that has at least one advantage over known devices for determining EBT.

### SUMMARY

The invention, in some embodiments, relates to methods and devices suitable for determining exhaled breath temperature.

According to an aspect of some embodiments of the teachings herein, there is provided a device suitable for determining exhaled breath temperature (EBT), comprising:
a conduit defining an air passage;
inside the air passage a heat collector;
a mouthpiece functionally-associated with the conduit and configured so that when a person exhales air through the mouth (typically, but not necessarily, while encircling the mouthpiece with the lips), the mouthpiece directs the exhaled air into the air passage, so that the exhaled air can exchange heat with the heat collector;
a temperature determiner configured to determine a temperature of the heat collector at least once during an exhalation through the mouthpiece; and
a controller comprising a computer processor and memory for receiving a temperature of the heat collector determined by the temperature determiner and for subsequently calculating an indicative temperature that is indicative of the temperature of the exhaled air, the calculating based on the temperature of the heat collector as determined by the temperature determiner.
   In accordance with the teachings herein, the indicative temperature is taken to be the EBT As used herein, the phrase “the exhaled air can exchange heat with the heat collector” and variants thereof means that the device is configured so that the heat collector heats-up or cools-down when there is a temperature difference between the heat collector and the exhaled air that flows through the air passage.

In some embodiments, the controller is configured so that the indicative temperature of the exhaled air is calculated to be the same as the temperature of the heat collector as determined by the heat determiner, that is to say, it is accepted that the temperature of the exhaled air (the EBT) is substantially the same as that of the heat collector. In such embodiments, it is recognized that the heat collector temperature as determined by the temperature determiner is not exactly the real temperature of the exhaled air, but is sufficiently close to be diagnostically useful, especially when individual measurements are compared one to the other and/or processed by the processor, providing, for example, the average value or trimmed mean value of the measured EBTs. In some embodiment, the controller is configured to calculate the indicative temperature of the exhaled air from the temperature of the heat collector as determined by the heat determiner but corrected for other factors, for example to account for the humidity of the exhaled air and/or is modified by a fudge factor to account for known and/or expected inaccuracies.

In some embodiments, the device further comprises a display functionally-associated with the controller to display the determined temperature and/or the calculated indicative temperature of the exhaled air. A display is any suitable component for displaying the determined temperature and/or the indicative temperature in a manner perceivable to a human, for example, an LED, LCD or plasma display screen that is physically or wirelessly associated with the controller, for example, the display screen of a suitably-configured smartphone, tablet or computer or a display screen that is an integral part of the device.

In some embodiments, the device further comprises a memory functionally-associated with the controller to store the determined temperature and/or the calculated indicative temperature. A memory is any suitable component for storing data as known in the art of computers, for example magnetic or electronic memory, for example, the memory of a suitably-configured smartphone, tablet or computer or a memory that is an integral part of the device.

In some embodiments, the device further comprises a transmitter (as used herein, the term “transmitter” includes a “transceiver”) functionally-associated with the controller to transmit the determined temperature and/or the calculated indicative temperature. Any suitable transmitter can be used, wired or wireless, for example, an infrared transmitter, Li-Fi or a radio-frequency transmitter such as telephony (of any suitable generation, e.g., 4G, 5G, LTE, LoRa, DASH7) Wi-Fi®, Bluetooth®, NFC, Zigbee. Such a transmitter is typically configured to transmit to a remote device such as a suitably-configured device such as a smartphone, tablet, computer or to a cloud.

In some embodiments, the device is configured to display and/or to store and/or to transmit to a remote device additional information, for example air humidity (in some embodiments where the device comprises a component to determine humidity in the air passage), air pressure (in some embodiments where the device comprises a pressure sensor to determine the air pressure inside the air passage), ambient air temperature (in some embodiments where the device comprises a component to determine ambient air temperature, flow rate (in some embodiments where the device comprises a flow detector to determine flow rate through the air passage) or other such information.

In some embodiments, the temperature determiner is configured to determine a temperature of the heat collector multiple times during a single exhalation through the mouthpiece. In some such embodiments, the temperature determiner is configured to determine a temperature of the heat collector at least once every two seconds, at least once a second and even at least twice a second. In preferred such embodiments, the controller is configured to calculate respective indicative temperatures for multiple received determined temperatures. In even more preferred such embodiments, the device is configured to display and/or to store and/or to transmit the multiple determined temperatures and/or indicative temperatures in order (i.e., any manner that allows identifying for a give temperature, a preceding temperature and a succeeding temperature, for example, storing the multiple determined temperatures and/or multiple indicative temperatures as a series, as an array, or associated/labeled with a serial number). In some embodiments where a device comprises a component to determine the starting time of an exhalation, a given indicative temperature or determined temperature is display and/or stored and/or transmitted associated with a time stamp indicating the time after the start of the exhalation that the corresponding indicative temperature was determined. In such embodiments, each such determined and/or indicative temperature corresponds to the determined and/or indicative temperature of the exhaled air at a different phase of the exhalation. In some embodiments, the EBT is measured multiple times per each phase of the exhalation.

It is known that when a person begins an exhalation, the first portion of the air that is exhaled from the mouth, into the mouthpiece and the air passage is air that is present in the oral cavity, followed by air from the larynx, from the trachea and then from the lungs, progressively from the more proximal parts of the lungs to the more distal parts of the lungs. Embodiments such as those described immediately herein above, where a temperature of the heat collector is determined multiple times during an exhalation, allow identification of the temperatures of different portions of the exhaled air. In some instances, identification of the temperature of different portions of the exhaled air is diagnostically useful, for example, may be indicative of the medical condition of a correspondent part of the subject's respiratory system.

In some embodiments, the temperature determiner and the controller are configured to determine a temperature of the heat collector for predefined or for a variable number of phases of a single exhalation or of multiple exhalations.

In some embodiments, the device comprises:
a conduit assembly that includes at least the conduit and the heat collector; and
a body assembly that includes other components of the device,
   wherein the conduit assembly and the body assembly are reversibly-separable from a coupled state in which the device can be used to determine EBT to a separated state. In such embodiments the conduit assembly and the body assembly can be separated to the separated state or coupled to the coupled state, allowing, for example, simple cleaning or replacement of a used conduit assembly. In some such embodiments, the mouthpiece is a component of the body assembly. Alternatively, in some such embodiments, the mouthpiece is a component of the conduit assembly.

In some embodiments, the mouthpiece is integrally-formed with the conduit

In some embodiments, the heat collector is physically connected to the conduit.

In some embodiments, the inner walls of the conduit in proximity to the heat collector have a thermal conductivity of not greater than about 0.5 W/(m K).

The dimensions and shape of the mouthpiece are any suitable dimensions and shape that allow a human to exhale breath through the mouth into the mouthpiece. Such dimensions and shapes are well-known in various fields including the fields of musical instruments and pulmonary medical devices. Specific known mouthpieces include mouthpieces of devices described in U.S. Pat. No. 11,719,101 and in WO 2023/081489.

The dimensions and shape of the air passage are any suitable dimensions and typically defined by the inner walls of the conduit.

In a longitudinal direction, the shape of the air passage is any suitable shape, including J-shaped, longitudinally curved and straight.

The proximal portion of the air passage, from the mouthpiece where the exhaled breath enters the air passage to the distal end of the heat collector, preferably has a constant cross-sectional area and shape. The cross-sectional shape of the air passage is preferably devoid of any vertex, for example, a circle, an oval, an ovoid. In preferred embodiments, the proximal portion of the air passage is essentially a straight cylinder having a cross sectional shape is preferably devoid of any vertex, preferably a circle.

The cross-sectional area of the proximal portion of the air passage is any suitable area. In preferred embodiments the cross-sectional area of the proximal portion of the air passage is at least about 0.2 cm² (for example, a circular cross section having a diameter of 0.5 cm) and more preferably at least about 0.8 cm² (for example, a circular cross section having a diameter of 1 cm). In preferred embodiments the cross-sectional area of the proximal portion of the air passage is not more than about 12.6 cm² (for example, a circular cross section having a diameter of 4 cm) and more preferably not more than about 3.2 cm² (for example, a circular cross section having a diameter of 2 cm).

The length of the proximal portion of the air passage is any suitable length and is typically determined so that the size of the device is convenient for human use. In some preferred embodiments, the length of the proximal portion of the air passage is not less than about 1 cm and even not less than about 2 cm. In some preferred embodiments, the length of the proximal portion of the air passage is not more than about 15 cm and even not more than 15 about 10 cm.

The cross-sectional shape of the distal portion of the air passage that is downstream of the distal end of the heat collector is any suitable shape, although typically for simplified manufacture the cross-sectional shape of the distal portion of the air passage is substantially the same as that of the proximal portion.

The cross-sectional size of the distal portion of the air passage is any suitable size, but preferably the cross-sectional size of the distal portion of the air passage is about the same or larger than that of the proximal portion of the air passage.

The length of the distal portion of the air passage is any suitable length and is typically determined so that the size of the device is convenient for human use and also to protect the heat collector from exposure to external factors that potentially affect the temperature measurements thereof. In some preferred embodiments, the length of the distal portion of the air passage is not less than about 1 cm and even not less than about 2 cm. In some preferred embodiments, the length of the distal portion of the air passage is not more than about 15 cm and even not more than about 10 cm. As noted herein below, in some embodiments the distal portion of the air passage includes a one-way valve to prevent backflow of air towards the heat collector.

In some embodiments, the heat collector has a 3-dimensional shape. In some such embodiments, the heat collector is hollow having an inner volume. In some such embodiments, the device comprises at least one hole (in preferred embodiments, multiple holes) in a surface of the heat collector that allow exhaled air to enter an inner volume of the heat collector. Additionally, or alternatively, in some embodiments inside the inner volume of the heat collector are ribs of a heat conductive material allowing quick heat transfer in the entire heat collector, helping to ensure that the different parts of the heat collector have the same temperature.

In some embodiments, the heat collector comprises a thin sheet. In some embodiments, the heat collector is a thin sheet.

In some embodiments, the heat collector comprises a wire. In some embodiments, the heat collector is a wire. In some embodiments, the wire is shaped into a 3D shape.

In some embodiments, the device further comprises a humidity sensor configured to determine a humidity of exhaled air flowing through the conduit, and to provide a determined humidity to the controller. Any suitable known humidity sensor may be used for 15 implementing such an embodiment. In some embodiments, the controller is configured to use a humidity of exhaled air received from the humidity sensor together with a corresponding temperature determined by the temperature determiner for the heat collector to calculate an indicative temperature to account for the humidity of the exhaled air. Determining the temperature of a gas by modifying a measured gas temperature to account for the humidity of the gas is well known to a person having ordinary skill in the art.

In some embodiments, the device further comprises a pressure sensor configured to determine the pressure of exhaled air in the air passage and to provide a determined pressure to the controller. Any suitable pressure sensor may be used for implementing such an embodiment.

In some embodiments, the device further comprises a flow detector configured to detect air flow through the air passage and to provide the controller with an indication that air is flowing in the air passage. Any suitable flow detector may be used for implementing such an embodiment, including flow detectors such as described in U.S. Pat. No. 11,179,101 (by one of the Inventors) and PCT publication WO 2023/081489 (by the Applicant). In some such embodiments, the flow detector is configured to determine a rate of air flow through the air passage and, in some such embodiments, to provide a determined rate of air flow to the controller. In some embodiments, the air flow detector is configured to determine air flow parameters and to provide the determined parameters to the controller.

In some embodiments, the device further comprises a component to determine when air starts to be exhaled into the air passage and to provide the controller with an indication that air is starting to be exhaled into the air passage. In some such embodiments, such a component is a pressure sensor and/or a flow detector and/or the temperature determiner and/or other component.

In some embodiments, the device further comprises a component to determine when air is being exhaled into the air passage and to provide the controller with an indication that air is being exhaled into the air passage. In some such embodiments, such a component is a pressure sensor and/or a flow detector and/or other component.

In some embodiments, the device further comprises an ambient air temperature determiner to determine the temperature of the ambient air, i.e., at the outside of the device. In some embodiments, the ambient air temperature determiner is configured to provide a determined ambient temperature to the controller. Any suitable temperature determiner may be used for implementing such an embodiment, for example a suitable thermometer.

In some embodiments, the device is configured to prevent backflow of air from the air passage past the heat collector. Specifically, such configuration ensures that ambient air or exhaled air that has already passed the heat collector does not flow back past the heat collector. Such backflow could change the temperature of the heat collector and thereby potentially distort the temperature measurements. In some such embodiments, the device comprises a one-way valve that prevents backflow of air from the air passage past the heat collector.

In some embodiments, the device further comprises a transmitter (in some embodiments a transmitter without being a receiver and in preferred embodiments a transceiver) configured to communicate with a remote device, wherein the transmitter is selected from the group consisting of a wired transceiver, an infrared transceiver, a radio-frequency transceiver and a Li-Fi transceiver.

In some embodiments, the device further comprises hardware and software/firmware configured to provide self-calibration of temperature measurements.

### Device Having Physical Connection Between Heat Collector and Temperature Determiner (Contact Temperature Measurement)

In some embodiments, the heat collector is in physical contact with a temperature sensor which is a component of the temperature determiner, the physical contact providing thermal conduction between the heat collector and the temperature sensor. In some such embodiments, the temperature sensor comprises a component selected from the group consisting of a thermocouple, a thermistor and an integrated circuit.

In some embodiments, the temperature sensor is in wired electrical communication with an electronic circuit which is a component of the temperature determiner that determines a temperature of the heat collector from information received from the temperature sensor through the wired communication. In some embodiments, the electronic circuit is a component of the controller. Alternatively, in some embodiments, the electronic circuit is a component separate from the controller but is functionally-associated therewith.

In some embodiments, the device comprises:
a conduit assembly that includes the conduit, the heat collector and the temperature sensor; and
a body assembly that includes the electronic circuit (and preferably also the controller),
   wherein the conduit assembly and the body assembly are reversibly-separable from a coupled state where there is wired communication between the temperature sensor and the electronic circuit, so that the device can be used to determine EBT to a separated state, where there is no wired communication between the temperature sensor and the electronic circuit.

### Device Having No Physical Connection Between Heat Collector and Temperature Determiner (Non-Contact Temperature Measurement)

In some embodiments, the temperature determiner comprises a non-contact thermometer having a detection window, the detection window positioned to receive IR radiation emitted from a location in the air passage where the heat collector is located when exhaled air is passing through the air passage.

In some embodiments, the detection window of the non-contact thermometer is located outside of the air passage. In such embodiments, typically the entire thermometer is located outside the air passage. In some such embodiments, at least a portion of the conduit is transparent to IR radiation, so that during an exhalation into the mouthpiece, IR radiation emitted from the heat collector passes through the IR-transparent portion of the conduit to be received by the detection window of the non-contact thermometer.

In some embodiments, the portion of the conduit that is transparent to IR radiation is smaller than the entire conduit, e.g., is an IR-transparent window.

In some embodiments, substantially the entire conduit is transparent to IR radiation.

In some embodiments, the device comprises:
a conduit assembly that includes the conduit and the heat collector; and
a body assembly that includes the non-contact thermometer (and preferably also the controller),
   wherein the conduit assembly and the body assembly are reversibly separable from a coupled state where the detection window of the non-contact thermometer is positioned to receive IR radiation emitted from a location in the air passage where the heat collector is located when exhaled air passes through the air passage, so that the device can be used to determine EBT, and a separated state.

In some embodiments, the heat collector is fixedly mounted inside the air passage.

In some alternative embodiments, the heat collector can move inside the air passage but, when air is exhaled into the air passage through the mouthpiece, the heat collector is found in a location in the air passage that allows IR radiation from the heat collector to enter the detection window of the non-contact thermometer. One embodiment of such a device is a variant of the device depicted in FIGS. 1A and 1B of PCT publication WO 2023/081489 by the Applicant which is included by reference herein at least for the purpose of supporting these embodiments. In the device, a movable component 28 is ordinarily in a first position but, when a user exhales into the mouthpiece of the device, the exhaled air moves movable component 28 to a second position. In the variant device which is an embodiment of the device according to the teachings herein, the movable component is the heat collector, for example, a ball of a metal such as aluminum, silver, copper or gold, preferably a hollow ball, preferably with heat-conductive chords passing in the hollow from one side to the other side of the ball and an IR-transparent detection window of a non-contact thermometer is positioned to receive IR radiation emitted from the location in the air passage where the heat collector is located when exhaled air is passing through the air passage, which is the second position of the heat collector.

According to an aspect of some embodiments of the teachings herein, there is also provided a method for determining exhaled breath temperature (EBT), the method comprising:
a. providing a device comprising:
   a conduit defining an air passage in which is located a heat collector, and
   a mouthpiece in fluid communication with the air passage (for example, any suitable embodiment of the device according to the teachings herein);
b. during an exhalation of air from the mouth of a subject through the mouthpiece and into the air passage during which the exhaled air exchanges heat with the heat collector (so that the heat collector heats up or cools down, depending on the relative temperatures of the exhaled air and the heat collector), determining the temperature of the heat collector at least one time during the exhalation; and
c. calculating an indicative temperature that is indicative of the temperature of the exhaled air, the calculating based on the temperature of the heat collector as determined by the temperature determiner.

As described above with referenced to the device of the teachings herein, the indicative temperature is taken to be the EBT.

In some embodiments, the method further comprises determining the humidity and/or pressure of the exhaled air and, during ‘c’, the indicative temperature is corrected for other factors such as to account for the humidity of the exhaled air and/or is modified by a fudge factor to account for known and/or expected inaccuracies.

In some embodiments, the method further comprises at least one of recording (e.g., on electronic or magnetic media), transmitting (wired or wirelessly) and displaying (in a manner perceivable to a human) the temperature and/or the measured humidity and/or pressure and/or parameters of the air flow of the exhaled air, and/or other relevant information.

In some embodiments, the temperature of the heat collector is determined multiple times during a single exhalation, in some embodiments, at a rate of not less than once every 2 seconds, in some embodiments at a rate of not less than once a second and in some embodiments at a rate of not less than twice a second.

Aspects and embodiments of the invention are described in the specification herein below and in the appended claims.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIGS. 1A and 1B schematically depict an embodiment of a device according to the teachings herein in side cross section; and

FIGS. 2A and 2B schematically depict another embodiment of a device according to the teachings herein in side cross section; and

FIG. 3 schematically depicts determination of the temperature of the heat collector at different phases of a single exhalation.

### DETAILED DESCRIPTION

Unless otherwise defined, all technical and scientific terms used herein have the same meaning as commonly understood by one of ordinary skill in the art to which the invention pertains. In case of conflict, the specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof. As used herein, the indefinite articles “a” and “an” mean “at least one” or “one or more” unless the context clearly dictates otherwise.

As used herein, when a numerical value is preceded by the term “about”, the term “about” is intended to indicate +/−10%. As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

The invention, in some embodiments, relates to methods and devices for determining exhaled breath temperature.

The principles, uses and implementations of the teachings of the invention may be better understood with reference to the accompanying description and figures. Upon perusal of the description and figures present herein, one skilled in the art is able to implement the teachings of the invention without undue effort or experimentation. In the figures, like reference numerals refer to like parts throughout.

Before explaining at least one embodiment of the invention in detail, it is to be understood that the invention is not limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

As noted in the introduction, it would be useful to have methods and devices for determining exhaled breath temperature. The teachings herein provide methods and devices that comprise a conduit defining an air passage in which is located a heat collector.

A subject, such as a human, exhales air from the respiratory system through the mouth into the air passage through a mouthpiece of the conduit.

The exhaled air flows through the air passage and heats up or cools down the heat collector, depending on the difference in temperature between the exhaled breath and the heat collector.

The temperature of the heat collector is determined. The determined heat collector temperature is, by inference, taken to be identical to or indicative of the temperature of the exhaled air or of a certain portion of the exhaled air related to a certain phase of an exhalation.

### Determining Temperature Multiple Times

In some embodiments, the temperature of the heat collector is determined multiple times per exhalation. In some such embodiments, the temperatures measured at different times during an exhalation are correlated with the temperatures of different parts of the respiratory system.

In some such embodiments, the temperature of the heat collector is determined at least once every two seconds, at least once a second and even at least twice a second.

In some alternate embodiments, different heat-collector temperatures are determined during multiple exhalations, where each exhalation is divided into multiple phases, and the temperature is determined separately for each phase of each exhalation (for example, T₁(a), T₁(b), T₁(c), T₁(d), T₁(e); T₂(a), T₂(b), T₂(c), T₂(d), T₂(e); T₃(a), T₃(b), T₃(c), T₃(d), T₃(e); where T₁(a) is the temperature determined within phase (a) of the first exhalation, T₂(a) is the temperature determined within the same phase (a) of the second exhalation, T₃(b) is the temperature determined within the next phase (b) of the third exhalation, etc.). In such way, for each phase (a), (b), . . . (i) of the exhalation an average temperature or trimmed mean value may be determined, which might be better correlated with the temperatures of different parts of the respiratory system. In some preferred embodiments, the beginning of each exhalation is taken as the start time to, and each phase is defined with a start time and an end time with relation to start time to. For example, in the above example, for each exhalation phase (a) can be defined as t₀+0 sec to t₀+0.5 sec, phase (b) defined as t₀+0.5 sec to t₀+1.0 sec, phase (c) defined as t₀+1.0 sec to t₀+1.5 sec, phase (d) defined as t₀+1.5 sec to t₀+2.0 sec, and phase (e) defined as t₀+2.0 sec to t₀+2.5 sec.

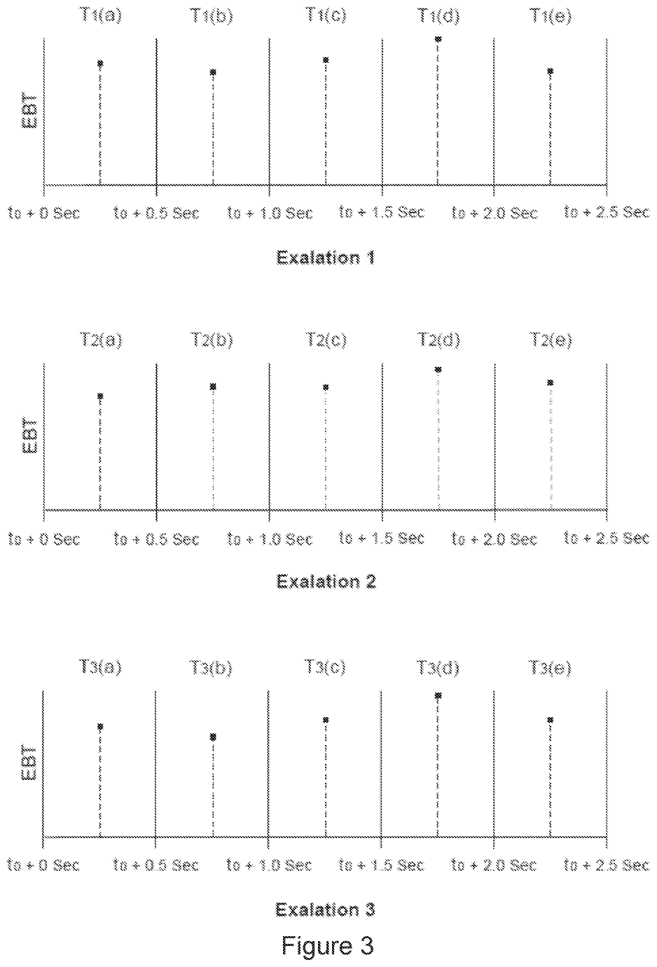

As an example, in FIG. 3 the phases (a), (b), (c), (d) and (e) of 3 successive exhalations Exhalation 1, Exhalation 2 and Exhalation 3 and the correspondent temperatures T₁(a), T₁(b), T₁(c), T₁(d), T₁(e); T₂(a) . . . T₃(e) are depicted. The duration of each of the phases (a), (b), . . . (i) of the exhalation is not necessarily the same, for example, phase (a) can be defined as t₀+0 sec to t₀+0.4 sec, phase (b) defined as t₀+0.4 sec to t₀+0.9 sec, phase (c) defined as t₀+0.9 sec to t₀+1.6 sec, phase (d) defined as t₀+1.6 sec to t₀+2.2 sec, and phase (e) defined as t₀+2.2 sec to t₀+3.0 sec.

In some embodiments, the EBT is measured multiple times per each phase of the exhalation.

Determination of the start time, duration of each phase and the end time of an exhalation may be easily implemented by one skilled in the art, based, for example, on detection of an exhaled air flow through the air passage, detection of a change in temperature of the heat collector (e.g., as determined by the temperature determiner) and/or detection of a change in the pressure in the air passage.

### Replaceable Conduit

In preferred embodiments, the device comprises a conduit assembly that includes at least the conduit and the heat collector (and, optionally, other components such as a temperature sensor (which is a component of the temperature determiner) and/or additional components, such as a humidity sensor and/or pressure sensor and/or air flow detector) and a body assembly that includes other components of the device. In preferred such embodiments, the conduit assembly and the body assembly are reversibly separable, e.g., reversibly user-separable, from a coupled state in which the device can be used to a separated state, and from the separated state to the coupled state. In some such embodiments the mouthpiece is a component of the body assembly but in some preferred embodiments the mouthpiece is a component of the conduit assembly.

In such embodiments, conduit assembly that includes components that during use of the device are exposed to contact with a subject's fluids (e.g., saliva and exhaled air) and therefore may be contaminated with pathogens, can be discarded, cleaned or sterilized, for example, by steam sterilization, while the body assembly that include only components that do not substantially contact a subject's fluids are retained for future use. In some embodiments the body assembly is configured to allow cleaning and sterilization using a suitable sterilization method, for example immersion sterilization.

In such embodiments, it is preferred that relatively expensive components of the device that are optionally present such as a display, a user interface, a processor, a wireless transmitter and/or a power supply such as a battery, are components of the body assembly. In some preferred embodiments the conduit assembly is configured to be disposable and comprises only relatively cheap components.

Heat Collector with Wired Connection Through the Conduit

In some embodiments, the heat collector is physically associated with the conduit and is preferably configured for efficient heat exchange with exhaled air in the air passage. In some such embodiments, the heat collector is in physical contact with a temperature sensor (which is a component of the temperature determiner), such as a thermocouple, a thermistor, an integrated circuit or other suitable temperature sensor, the physical contact providing thermal conductivity between the heat collector and the temperature sensor. The temperature sensor is preferably in wired electrical communication with an electronic circuit (which is a component of the temperature determiner) that determines the temperature of the heat collector from information received from the temperature sensor through the wired communication.

In preferred such embodiments, the conduit, the heat collector and the temperature sensor are part of a conduit assembly, and the electronic circuit is a part of a body assembly, the conduit assembly and the body assembly are reversibly separable from a coupled state, where there is wired communication between the temperature sensor and the electronic circuit, to a separated state, where there is no wired communication between the temperature sensor and the electronic circuit.

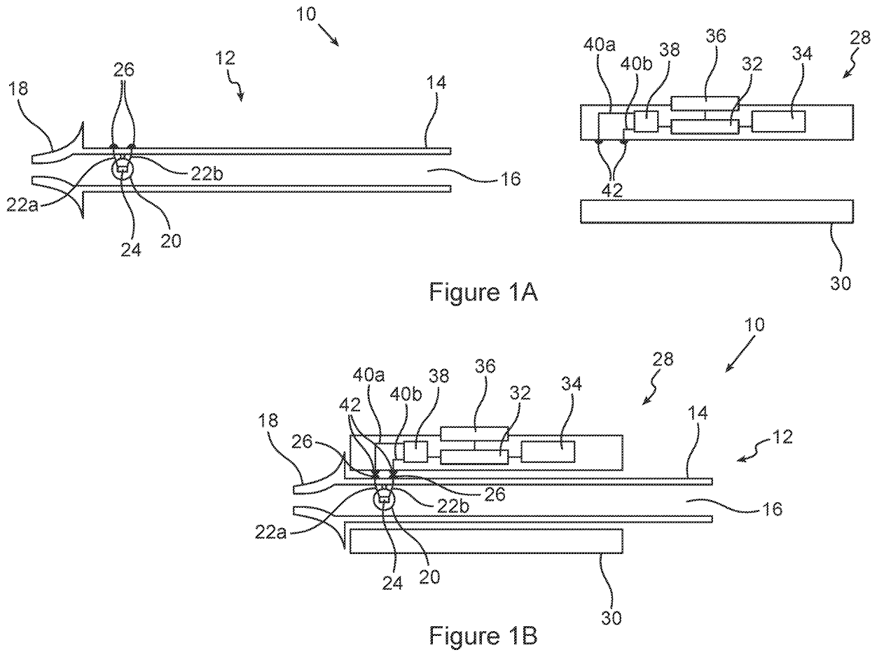

Such an embodiment is device 10 schematically depicted in FIGS. 1A and 1B, in side cross section.

Device 10 includes a conduit assembly 12 comprising a conduit 14 defining an air passage 16, conduit 14 having an integrally-formed mouthpiece 18 at a proximal end thereof and a heat collector 20 disposed in air passage 16. Heat collector 20 is physically associated with a temperature sensor 24 (such as thermistor, thermocouple, or integrated circuit) which has leads 22a and 22b that pass through the walls of conduit 14 and are connected to electrical contacts 26. In device 10, heat collector 20 is a crumpled sheet of thin copper foil that is tightly wrapped around temperature sensor 24.

Device 10 also includes a body assembly 28 comprising a body 30 made of plastic and a controller 32 (including a microprocessor on a printed circuit board) functionally associated with a power source 34 (a primary or rechargeable battery), a display screen 36 and a temperature measurement electronic circuit 38 having two conductive circuit leads 40a and 40b that are electrically connected to electrical contacts 42.

In FIG. 1A, device 10 is depicted in a separated state, where there is no wired electrical communication between temperature sensor 24 and temperature measurement circuit 38.

In FIG. 1B, device 10 is depicted in a coupled state where, inter alia, contacts 26 make electrical contact with contacts 42, thereby establishing wired communication between temperature sensor 24 and temperature measurement circuit 38.

When device 10 is in the coupled state of FIG. 1B, a person exhales air from the mouth through mouthpiece 18 into air passage 16, preferably while encircling mouthpiece 18 with the lips. During the exhalation, heat collector 20 heats up or cools down to approach and even attain the temperature of exhaled air that passes through air passage 16. The temperature of the temperature sensor 24 which is in thermal communication with heat collector 20 also changes. Controller 32 determines the temperature of heat collector 20 and, by inference, of the exhaled air from the data received from temperature sensor 24 by temperature measurement electronic circuit 38. The determined temperature is displayed by controller 32 on display screen 36 and/or transmitted to a remote device.

Heat Collector with Non-Contact Thermometer

In some embodiments, the temperature determiner of the device comprises a non-contact thermometer, the non-contact thermometer having an IR-transparent detection window that is positioned to receive IR radiation emitted from a location in the air passage where the heat collector is located when exhaled air is passing through the air passage. When exhaled air passes through the air passage, the heat collector heats up or cools down to approach and even attain the temperature of exhaled air that passes through the air passage. The heat collector emits IR radiation that is related to the temperature thereof, which IR radiation is received by the non-contact thermometer through the detection window allowing to determine the temperature of the heat collector and by inference of the exhaled air.

In preferred embodiments, the detection window of the non-contact thermometer is located outside of the air passage. In some such embodiments, at least a portion of the conduit is transparent to IR radiation, so that IR radiation emitted from the heat collector passes through the IR-transparent portion of the conduit to be received by the detection window of the non-contact thermometer.

In preferred such embodiments, the heat collector and the conduit are part of a conduit assembly and the non-contact thermometer (e.g., MLX90614 by Melexis NV, Ypres, Belgium) is a component of a body assembly, the conduit assembly and the body assembly are reversibly separable from the coupled state, where the detection window of the non-contact thermometer is positioned to receive IR radiation emitted from a location in the air passage where the heat collector is located when exhaled air passes through the air passage, and a separated state.

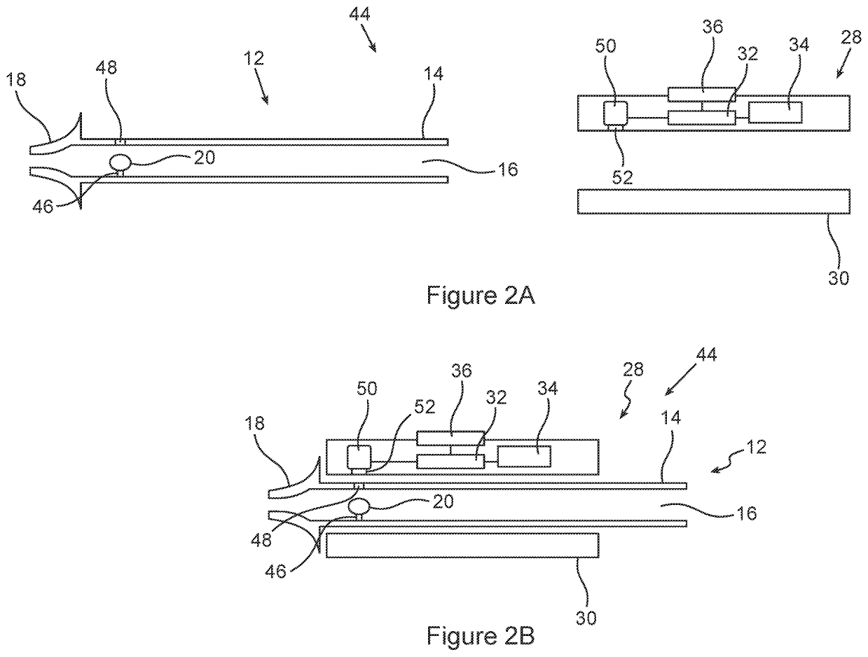

Such an embodiment is device 44 schematically depicted in FIGS. 2A and 2B, in side cross section.

Device 44 includes a conduit assembly 12 comprising a conduit 14 defining an air passage 16, conduit 14 having an integrally-formed mouthpiece 18 at a proximal end thereof and a heat collector 20 disposed in air passage 16. In device 44, heat collector 20 is a ball made of thin aluminum foil (as an example, with thickness of 0.3-0.5 mm) that is physically connected to conduit 14 with a holder 46 that anchors heat collector 20 in a fixed location in air passage 16. A portion of conduit 14 is an IR-transparent window 48 (e.g., of PMMA) in proximity to heat collector 20.

Device 44 also includes a body assembly 28 comprising a body 30 and a controller 32 (including a microprocessor on a printed circuit board) functionally associated with a power source 34 (a primary or rechargeable battery), a display screen 36 and a non-contact thermometer 50 having a detection window 52. Body 30 is made of opaque polycarbonate that completely blocks the passage of light (especially IR light).

In FIG. 2A, device 44 is depicted in the separated stated.

In FIG. 2B, device 44 is depicted in a coupled state where, inter alia, detection window 52 of non-contact thermometer 50 is positioned to receive IR radiation that is emitted from heat collector 20 and passes through IR-transparent window 48. Body 30 prevents any ambient IR radiation from reaching heat collector 20.

When device 44 is in the coupled state depicted in FIG. 2B, a person exhales air from the mouth through mouthpiece 18 into air passage 16. During the exhalation, heat collector 20 heats or cools to approach and even attain the temperature of exhaled air that passes through air passage 16. The temperature of heat collector 20, and therefore of the exhaled air, is determined by controller 32 and displayed on display screen 36 with reference to data received from non-contact thermometer 50.

In device 44, conduit 14 comprises IR-transparent window 48. Such an IR-transparent window is smaller than the entire conduit. In some alternate embodiments, substantially the entire conduit (e.g., at least about 70%, at least about 80% and even at least about 90% of the conduit) is transparent to IR radiation, e.g., the conduit is made of PMMA.

In device 44, heat collector 20 is fixedly mounted inside air passage 16 due to holder 46. In some embodiments, heat collector 20 can move inside air passage 16 but, when air is exhaled into air passage 16 through mouthpiece 18, it is found in a location in air passage 16 that allows IR radiation from heat collector 20 to enter detection window 52 of non-contact thermometer 50.

It is preferable that the heat collector has a relatively low heat capacity, so that the temperature of the heat collector surface changes relatively quickly during interaction with exhaled air. In some embodiments, the heat collector has a relatively low mass and/or has at least a portion made of a material having a low volumetric heat capacity. In some embodiments, at least part of the heat collector is made of a material that has a molar heat capacity of not more than about 30 J/(mol K) and even not more than about 15 J/(mol K). In some embodiments, at least about 30% by weight of the heat collector is made of a material that has a molar heat capacity of not more than about 30 J/(mol K) and even not more than about 15 J/(mol K). In some embodiments, at least about 50% by weight of the heat collector is made of a material that has a molar heat capacity of not more than about 30 J/(mol K) and even not more than about 15 J/(mol K).

It is also preferable that the heat collector has a relatively high thermal conductivity so that the temperature of the entire heat collector surface will equalize relatively quickly during interaction with exhaled air. In some embodiments, at least part of the heat collector is made of a material that has a thermal conductivity that is not less than about 20 W/m K and even not less than about 100 W/m K, and even not less than about 300 W/m K. In some embodiments, at least about 30% by weight of the heat collector is made of a material that has a thermal conductivity that is not less than about 20 W/m K and even not less than about 100 W/m K, and even not less than about 300 W/m K. In some embodiments, at least about 50% by weight of the heat collector is made of a material that has a thermal conductivity that is not less than about 20 W/m K and even not less than about 100 W/m K, and even not less than about 300 W/m K.

It is also preferable that the heat collector has a relatively high emissivity, in particular, in the IR part of the spectrum. In some embodiments, at least part of the heat collector is made of a material that has an emissivity that is not less than about 0.05 and even not less than about 0.1. In some embodiments, at least about 30% by weight of the heat collector is made of a material that has an emissivity that is not less than about 0.05 and even not less than about 0.1. In some embodiments, at least about 50% by weight of the heat collector is made of a material that has an emissivity that is not less than about 0.05 and even not less than about 0.1.

Materials suitable for a heat collector include, but are not limited to:

| | thermal conductivity | molar heat capacity | |
|---|---|---|---|
| material | [W/m K] | [J/(mol K)] | emissivity |
| alumiplate Al | 20 | 24.2 | 0.1-0.25 |
| anodized Al | 20 | 24.2 | 0.6-0.95 |
| Au | 314 | 25.42 | 0.05 |
| Cu | 385 | 24.47 | 0.1 |
| Ag | 406 | 24.9 | 0.05 |
| SiC | 495 | | 0.8-0.95 |
| graphite | 25-470 | 10 | 1 |
| diamond | 1000 | 6.12 | 1 |
| graphene | 1000-2000 | 10 | 1 |
| black soot | 0.07 | 10 | 1 |

In device 44, heat collector 20 is a sphere made of thin aluminum foil (for example, having 0.3 mm to 0.5 mm thickness). In some embodiments, a heat collector has a 3-dimensional shape, for example, a sphere, a teardrop shape, a bullet shape, olive-shape, and cubic. In preferred embodiments, such a 3-dimensional shape is hollow. An advantage of a hollow 3-dimensional shape is that the heat capacity of the heat collector is reduced yet the surface area to interact with exhaled air in the air passage is relatively large.

In some embodiments, a heat collector is hollow, having an empty inner volume. In some such embodiments, the walls of the heat collector are not more than about 1 mm thick, not more than about 0.5 mm and even not more than about 0.3 mm thick. In some such embodiments, the walls of the heat collector are not less than about 0.05 mm thick and even not less than about 0.01 mm thick. In preferred such embodiments, there are holes in the surface of the heat collector which allow the exhaled air to enter the inner volume of the heat collector and heat the heat collector from the inside.

In some embodiments, a heat collector is hollow and inside the inner volume of the heat collector are ribs of a heat conductive material, allowing quick heat transfer within the entire heat collector which allows faster temperature equalization of the entire surface of the heat collector. For example, in some such embodiments the heat collector is a hollow aluminum sphere with aluminum chords spanning the inner volume, prepared for example by 3D printing.

In some alternate embodiments, the heat collector at least partially comprises, and in some embodiments is a thin sheet (e.g., 2-dimensional shape such as a sheet, a plate, a foil a film) for example, in some embodiments, the heat collector is a sheet of foil. In some such embodiments, the heat collector is not more than about 2 mm thick, not more than 1 mm thick, not more than about 0.5 mm and even not more than about 0.3 mm thick. In some such embodiments, the heat collector is not less than about 0.1 mm thick, not less than about 0.05 mm thick and even not less than about 0.01 mm thick.

In some embodiments, the heat collector is aerodynamically shaped to minimize drag and reduce the formation of turbulence in exhaled air flowing in the air passage. Alternatively, in some embodiments the heat collector has a surface that increases formation of turbulence near the surface allowing an increased volume of air to interact with the heat collector, for example, the heat collector has a rough surface that includes fins, protuberances, spikes and the like.

In some embodiments, a heat collector at least partially comprises, and in some embodiments is a wire (e.g., of aluminum, copper, gold, silver and alloys thereof). In some preferred such embodiments, the wire is shaped (e.g., one or more of folded, bent, crumpled, woven, wound) into a 3D shape, most preferably shaped into a hollow 3D shape. As used herein, “wire” means any elongated component having a length dimension that is at least times longer than an axial dimension and includes components such as ribbons, strands, filaments, wire, thread, rod, string and the like. In some such embodiments, the wire has a radial dimension (e.g., diameter) of not more than about 2 mm, not more than about 1 mm, not more than about 0.6 mm and even not more than about 0.3 mm. Additionally or alternatively, in some embodiments the wire has a radial dimension of not less than about 0.05 mm and even not less than about 0.01 mm.

In some embodiments, a heat collector comprises a coating (in some such embodiments not less than about 1 micrometer and even not less than about 5 micrometer; additionally or alternatively, not more than about 500 micrometers, e.g., 200-300 micrometers, of a material having a high thermal conductivity e.g., not less than about 100 W/m K (such as graphene, graphite, diamond, SiC, gold, copper, silver and alloys thereof) and/or low volumetric heat capacity material supported by a core of material having a low thermal conductivity and/or high volumetric heat capacity. Such a heat collector can be made by any suitable method, e.g., plating or deposition.

In devices 10 and 44, mouthpiece 18 is integrally formed with conduit 14. In some embodiments, a mouthpiece is a component separate from the conduit and is attached thereto. In some such embodiments, the mouthpiece is a component of the body assembly. Preferably, the mouthpiece is a component of the conduit assembly.

The inner walls of the conduit (that line the air passage) are of any suitable material having any suitable property. In preferred embodiments, the inner walls of the conduit (especially in the proximity of the heat collector) have a low thermal conductivity and/or are properly shaped and/or have appropriate surface to facilitate a laminar air flow near the walls, so that little, preferably no, heat is exchanged between the walls of the conduit and exhaled air flowing therethrough. In some embodiments, the inner conduit walls (especially in the proximity of the heat collector) have a thermal conductivity of not greater than about 0.5 W/(m K). In some embodiments, the inner conduit walls in proximity of the heat collector comprise at least 50% by weight of a material selected from the group consisting of plastics, PVC (polyvinyl chloride), polycarbonate, PP (polypropylene), PET (polyethylene terephthalate), PE (polyethylene), polyamide (Nylon) and PTFE (polytetrafluoroethylene).

In some embodiments of the device, for example variants of devices 10 or 44, a controller 32 includes or is functionally associated with additional components such as a computer memory, a wireless transmitter or transceiver, an on/off switch and other peripheral hardware as well as being software/firmware configured for operation of device 10 or 44.

In some embodiments, a device comprises a humidity and/or pressure sensor configured to determine the humidity and/or pressure of the exhaled air flowing through the conduit, and preferably configured to provide a determined humidity and/or pressure to the controller. In some such embodiments, the output of the humidity and/or pressure sensor is used (preferably by the controller) to adjust a calculation of the temperature of a heat collector and/or of the exhaled air. Specifically, the output of the humidity sensor is used (for example, by the controller) to correct a temperature of the heat collector as determined by the temperature determiner to account for the humidity of the exhaled air.

In some embodiments, a device is configured so that the inner walls of the conduit are insulated from the ambient temperature such as ambient air or the heat of a hand of a user holding the device.

In some embodiments, a device is configured to reduce, more preferably prevent, the inhalation of air through the air passage. Specifically, the device is configured to prevent backflow of air from the air passage through the mouthpiece, for example, as a result of a person inhaling through the mouth while the mouthpiece is held in the mouth. In some such embodiments, the conduit comprises a one-way valve. Such a one-way valve is useful to prevent the backflow of air from the air passage through the mouthpiece. Depending on the embodiment, a one-way valve is positioned at a distal end of the conduit, in the mouthpiece, or somewhere along the air passage. In preferred embodiments, a one-way valve is positioned downstream of the heat collector to reduce and even prevent the incidence of ambient air entering the air passage to influence the temperature of the heat collector.

In devices 10 and 44, controller 32 receives data related to the temperature of heat collector 20, calculates the temperature in a meaningful scale (e.g., ° C. or ° F.) and displays the temperature on display screen 36. In some alternative embodiments, the temperature of heat collector 20 is calculated from the data at a location remotely from the device, for example, the data is transmitted to a remote processor such as a smartphone processor which calculates the temperature in a meaningful scale. Additionally or alternatively, in some alternative embodiments, a calculated temperature is displayed at a location remotely from the device, for example, on a smartphone display screen. Additionally or alternatively, in some embodiments a calculated temperature is not displayed but data related to the temperature of a heat collector is used in a different way.

In some embodiments, additionally to determination of a momentary temperature of the heat collector, the controller is configured to implement additional features, such as: calculation of the average, trimmed mean value, the maximum and the minimum temperature values for predetermined or variable (user-controlled) number of breathes or time period (for example, 10 breathes or 30 seconds); graphical presentation of temperature variations over time; self-calibration, inter alia utilizing built-in NIST traceable High-Accuracy Digital Temperature Sensor (e.g., SHT35-DIS by Sensirion, Switzerland).

### Self-Calibration

Calibration of a non-contact IR thermometer is not an easy task and usually requires special calibration equipment such as blackbody cavity, precise reference thermometer, humidity meter, etc. Such calibration equipment is quite expensive and requires special skills to operate it properly.

In some embodiments, device 44 comprising a non-contact IR thermometer is configured to perform self-calibration. When the device is not used for a certain time, for example, for several hours, the temperature of all the elements inside the device, including the heat collector, becomes equal to the ambient temperature. Considering that the modern electronic components can operate in the micropower range, the influence of the electronic circuitry on the internal device temperature during self-calibration is negligeable. To ensure better temperature stability, the device may be kept in a thermally insulated container. The temperature of the heat collector is measured by IR thermometer of the device, and this temperature is compared to the temperature measured by a built-in High-Accuracy Digital Temperature Sensor (e.g., SHT35-DIS by Sensirion, Switzerland). The results of both measurements should be the same, and if they differ, the difference is used to calibrate the IR thermometer. To achieve even more precise calibration, before performing the self-calibration, the device should be kept for certain time in a thermostat at various ambient temperatures, for example, 15° C., 20° C., 25° C., 30° C. and 35° C. Calibration values determined for each temperature are stored in the memory and used during the EBT measurements.

In some embodiments, humidity of the air is measured additionally using a built-in humidity sensor and is further used for calibration of the non-contact IR thermometer.

In some embodiments, device 10 which comprises a contact thermometer is configured to perform self-calibration similarly to the self-calibration process described for device 44.

In devices 10 and 44, power source 34 is a battery (e.g., a primary or rechargeable cell). Embodiments of the device use any suitable power source, such as an ultracapacitor or solar cells.

1.-38. (canceled)

39. A device for determining exhaled breath temperature (EBT), comprising:
a conduit defining an air passage;
a heat collector disposed inside the air passage;
a mouthpiece functionally associated with the conduit and configured so that when a person exhales air through the person's mouth and into the mouthpiece, the mouthpiece directs the exhaled air into the air passage, so that the exhaled air can exchange heat with the heat collector;
a temperature determiner configured to determine a temperature of the heat collector at least once during an exhalation through the mouthpiece; and
a controller comprising a computer processor and memory for receiving the temperature determined by the temperature determiner and for subsequently calculating an indicative temperature that is indicative of a temperature of the exhaled air, the calculating based on the temperature determined by the temperature determiner.

40. The device of claim 39, wherein the conduit and the heat collector are comprised in a conduit assembly, and wherein the device further comprises:
a body assembly that comprises a plurality of other device components,
wherein the conduit assembly and the body assembly are reversibly separable from (i) a coupled state in which the device can be used to determine the EBT, to (ii) a separated state.

41. The device of claim 39, wherein the heat collector is physically connected to the conduit.

42. The device of claim 39, wherein the temperature determiner further comprises a temperature sensor, and wherein the heat collector is in physical contact with the temperature sensor, the physical contact providing thermal conduction between the heat collector and the temperature sensor.

43. The device of claim 39, wherein the temperature determiner comprises a non-contact thermometer having a detection window, the detection window positioned to receive infrared (IR) radiation emitted from a location in the air passage where the heat collector is located when the exhaled air is passing through the air passage.

44. The device of claim 43, wherein the detection window of the non-contact thermometer is located outside of the air passage.

45. The device of claim 44, wherein at least a portion of the conduit is transparent to the IR radiation, so that the IR radiation emitted from the heat collector passes through the IR-transparent portion of the conduit to be received by the detection window of the non-contact thermometer.

46. The device of claim 43, further comprising:
a conduit assembly comprising the conduit and the heat collector; and
a body assembly comprising the non-contact thermometer,
wherein the conduit assembly and the body assembly are reversibly separable from (i) a coupled state where the detection window of the non-contact thermometer is positioned to receive the IR radiation emitted from the location in the air passage, so that the device can be used to determine the EBT, and (ii) a separated state.

47. The device of claim 43, wherein the heat collector is fixedly mounted inside the air passage.

48. The device of claim 43, wherein the heat collector is configured to move inside the air passage, and wherein, when the air is exhaled into the air passage through the mouthpiece, the heat collector is positioned in the location in the air passage.

49. The device of claim 39, wherein inner walls of the conduit are in proximity to the heat collector, the inner walls having a thermal conductivity of not greater than about 0.5 Watts per meter-Kelvin (W/mK).

50. The device of claim 39, wherein the heat collector has a 3-dimensional shape.

51. The device of claim 39, wherein the heat collector comprises a thin sheet.

52. The device of claim 39, wherein the heat collector comprises a wire.

53. The device of claim 39, further comprising a pressure sensor configured to determine the pressure of the exhaled air flowing through the conduit, and to provide a determined pressure to the controller.

54. The device of claim 39, further comprising an air flow detector configured to detect air flow through the conduit and to provide the controller with an indication that air is flowing in the air passage.

55. The device of claim 39, configured to prevent backflow of the air from the air passage past the heat collector.

56. A method for determining exhaled breath temperature (EBT), the method comprising:
providing a device comprising:
a conduit defining an air passage,
a heat collector disposed inside the air passage, and
a mouthpiece in fluid communication with the air passage;
when air is exhaled from the mouth of a subject through the mouthpiece and into the air passage so that the exhaled air exchanges heat with the heat collector, determining, by the device, a temperature of the heat collector at least one time during the exhalation; and
calculating, by the device, an indicative temperature that is indicative of a temperature of the exhaled air, the calculating based on the temperature determined by the temperature determiner.

57. The method of claim 56, wherein the indicative temperature is either (i) corrected to account for the humidity of the exhaled air, and/or (ii) modified by a factor to account for one or more inaccuracies.

58. The method of claim 57, wherein the temperature of the heat collector is determined by the temperature determiner multiple times during the exhalation.
